(19) United States
(12) Patent Application Publication
(10) Pub. No.: US 2025/0272899 A1
(43) Pub. Date: Aug. 28, 2025

(54) APPARATUS AND METHOD FOR A NEURAL INTERFACE UTILIZING SENSORY COMMUNICATION TO AFFECT A BROAD SPECTRUM OF PHYSIOLOGIC ACTIVITY IN THE HUMAN BODY

(71) Applicant: Synapcis Inc., Mill Valley, CA (US)

(72) Inventors: Michael H. MOSKOWITZ, Mill Valley, CA (US); Glen WEST, Mill Valley, CA (US)

(21) Appl. No.: 19/088,669

(22) Filed: Mar. 24, 2025

Related U.S. Application Data
(63) Continuation-in-part of application No. 18/590,754, filed on Feb. 28, 2024, now abandoned.

(51) Int. Cl. G06T 11/60; G02B 27/01; G06T 3/04

(52) U.S. Cl. CPC G06T 11/60; G02B 27/0172; G06T 3/04; G02B 2027/0174

(57) ABSTRACT

A method includes projecting in a digital space an original object. The original object is transformed into a collection of disaggregated symbols displayed in the digital space. The collection of disaggregated symbols is aggregated into an aggregated object that has qualities or features in common with the original object. The aggregated object is presented in the digital space with auditory stimuli. All operations are performed without the use of any visual or auditory language.

### CROSS-REFERENCE TO RELATED APPLICATION

This is a continuation-in-part of U.S. Ser. No. 18/590,754, filed Feb. 28, 2024, the contents of which are incorporated herein by reference.

### FIELD OF THE INVENTION

The invention relates generally to the use of digital imagery to promote human wellbeing. More particularly, this invention is directed toward creating and displaying multiple symbolic stimuli to influence brain-body rhythmicity, decision-making processes, and to promote positive neuroplastic change.

### BACKGROUND OF THE INVENTION

Most individuals would like to steer the course of their own wellbeing and mental health based upon their personal belief system to direct positive outcomes, behaviors, emotions, and cognitions. Such outcomes are particularly desirable if achieved without the need for medical intervention. This would allow an individual to develop new habit formation, positive ideas, and thoughts, improved self-soothing, more effective self-agency, more nuanced problem solving, more productive executive function, and more effective and beneficial brain-body interaction.

### SUMMARY OF THE INVENTION

A method includes projecting in a digital space an original object. The original object is transformed into a collection of disaggregated symbols displayed in the digital space. The collection of disaggregated symbols is aggregated into an aggregated object that has qualities or features in common with the original object. The aggregated object is presented in the digital space with auditory stimuli. All operations are performed without the use of any visual or auditory language.

### BRIEF DESCRIPTION OF THE FIGURES

The invention is more fully appreciated in connection with the following detailed description taken in conjunction with the accompanying drawings, in which:

FIG. 1 illustrates a system configured in accordance with an embodiment of the invention.

FIG. 2 illustrates processing operations associated with an embodiment of the invention.

FIG. 3 illustrates symbolic stimuli displayed in accordance with an embodiment of the invention.

FIG. 4 illustrates symbol processing performed in accordance with an embodiment of the invention.

FIG. 5 illustrates executive function symbol stimuli utilized in accordance with an embodiment of the invention.

FIG. 6 illustrates problem solving symbol stimuli utilized in accordance with an embodiment of the invention.

FIG. 7 illustrates habit formation symbol stimuli utilized in accordance with an embodiment of the invention.

FIG. 8 illustrates planning symbol stimuli utilized in accordance with an embodiment of the invention.

FIG. 9 illustrates adjustment and adaptation symbol stimuli utilized in accordance with an embodiment of the invention.

Like reference numerals refer to corresponding parts throughout the several views of the drawings.

### DETAILED DESCRIPTION OF THE INVENTION

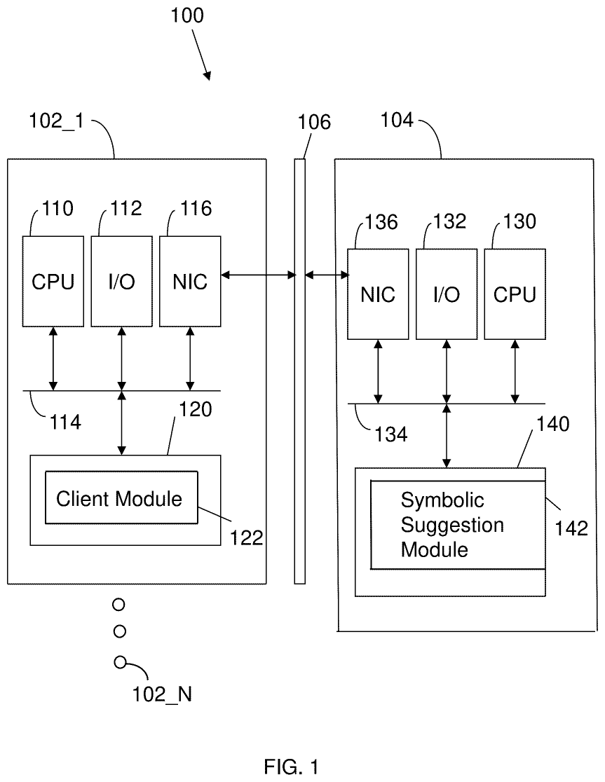

FIG. 1 illustrates a system 100 configured in accordance with an embodiment of the invention. The system 100 includes a set of client devices 102_1 through 102_N in communication with a server 104 via a network 106, which may be any combination of wired and wireless networks. The client devices 102_1 through 102_N may be computers, tablets, mobile devices, game consoles, game controllers, head-mounted devices and the like. By way of example, client device 102_1 includes a processor 110 (e.g., a central processing unit) in communication with input/output devices 112 via a bus 114. The input/output devices 112 may include input devices, such as a keyboard, mouse, touch display and the like. The output devices include at least one display, which may be circular. A circular display is a volumetric circular screen that projects images within a cylindrical volume. The output devices may include multiple display devices. The output devices may be head-mounted devices. A network interface circuit 116 is also connected to bus 114 to provide connectivity to network 106. A memory 120 is also connected to the bus 114. The memory 120 stores a client module 122 with instructions executed by processor 110 to access server 104 to obtain digital content of the type disclosed herein. The digital content is displayed to a user on one or more output devices.

Server 104 includes a processor 130, input/output devices 132, bus 134 and network interface circuit 136. A memory 140 is connected to bus 134. The memory 140 stores a symbolic suggestion module 142 with instructions executed by processor 130 to implement operations disclosed herein. The symbolic suggestion module 142 may be installed on any one of client devices 102_1 through 102_N. The invention is disclosed in the context of a client-server environment by way of example, not by way of limitation.

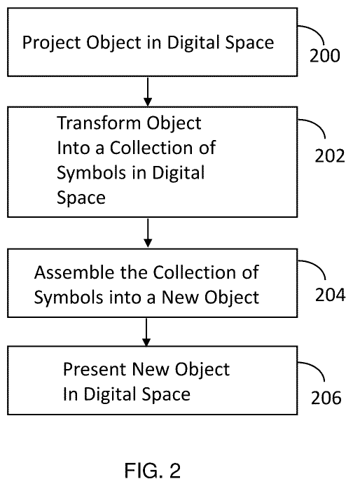

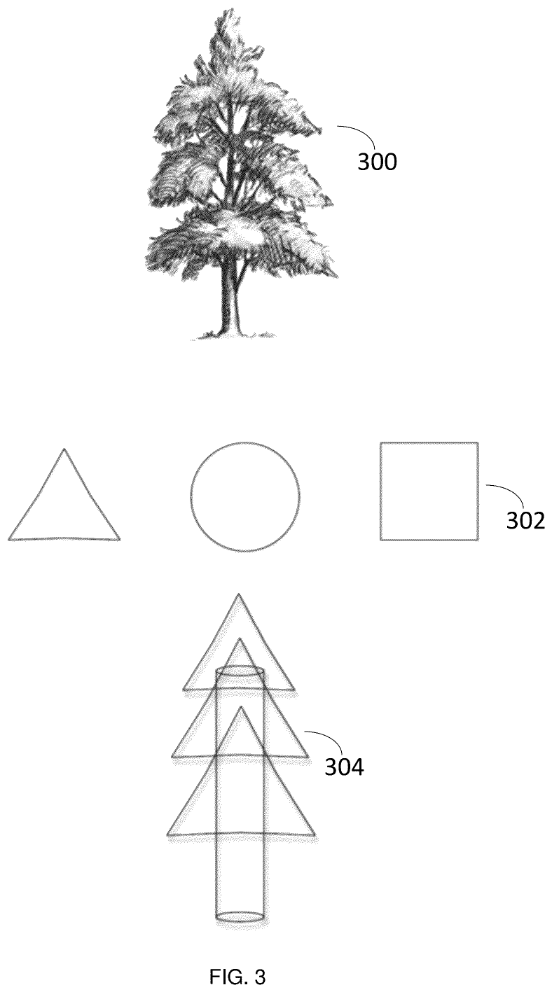

FIG. 2 illustrates operations implemented by the symbolic suggestion module 142. An original object is projected in a digital space 200. By way of example, FIG. 3 illustrates an original object 300 in the form of a tree which may be projected on a display, headset, a set of displays, etc. The original object is derived from the geometric components of an existing object that appears in the external environment (the natural world) or the internal environment (any object generated inside and representative of processes inside an individual's physical body). In the case of the internal environment, the invention utilizes a human brain analog. The human brain analog is manifested as executable code in the symbolic suggestion module 142. This executable code is referred to as a digital model of the human brain.

Returning to FIG. 2, the object is then transformed into a collection of symbols in the digital space 202. Returning to FIG. 3, a set of symbols 302 are projected in the digital space. The next operation in FIG. 2 is to assemble the collection of symbols into a new object that resembles the original object 204. The new object is then presented in the digital space 206. FIG. 3 illustrates a new object 304 that resembles the original object 300. The new object 304 comprises symbols from the symbol collection 302.

The user viewing this sequence of digital images is likely to elicit a pleasurable response to original object 300 and new object 304. The collection of symbols 302 is unlikely to elicit either a positive or a negative response.

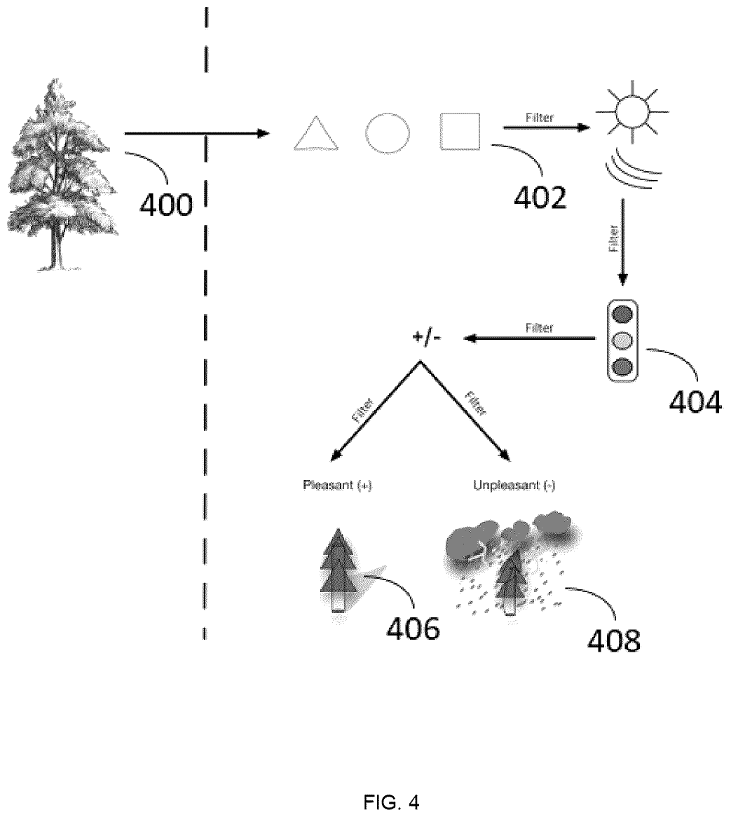

FIG. 4 illustrates an original object 400, a collection of symbols 402 derived from the original object and a filter 404 to alter the collection of symbols. Different plausible alterations are detailed below. The filter operation may result in an image 406 that induces a pleasurable user response or an image 408 that induces an unpleasant user response. The filter 404 is executable code associated with the symbolic suggestion module 142.

In this example, an object 400 in the natural world is transformed into the virtual world by breaking the components of the tree down into its basic geometry, resulting in a collection of symbols 402. This geometry is then filtered positively and negatively by context, risk, desirability and vallance of experience, resulting in an internal response in the digital model of the human brain to a targeted output.

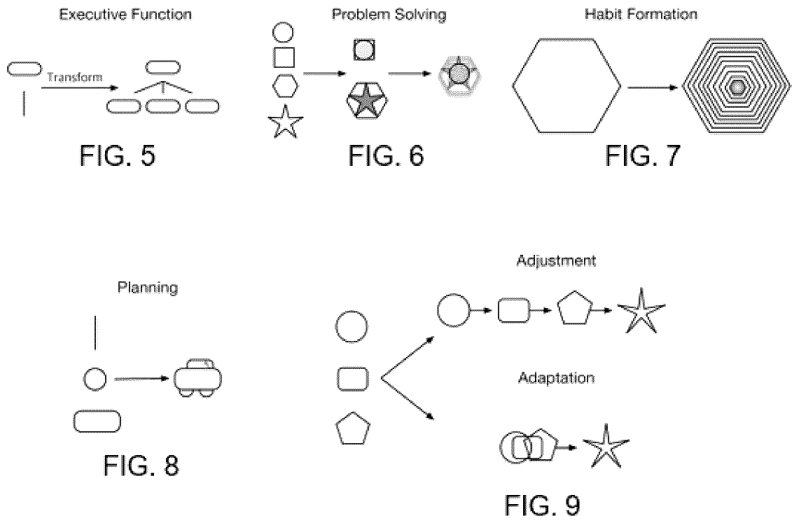

FIGS. 5-9 show how symbols extracted from an original object, in any environment, are altered to evoke predictable responses to a transformed virtual object in a digital space, including virtual reality, augmented reality, and mixed reality.

FIG. 5 shows static geometric shapes (a rectangle with rounded corners, line) that are transformed to visualize a symbolic, hierarchical (organizational) chart thereby invoking an executive function response in a user.

FIG. 6 illustrates static geometric shapes (circle, square, hexagon, star) that are unified into combined forms that become more complex, thereby invoking a problem-solving response in a user.

FIG. 7 shows a static geometric shape (hexagon) replicated and scaled uniformly to display persistent layering, resulting in a reinforced core that represents and evokes a habit formation response in a user.

FIG. 8 shows static geometric shapes (rectangle with rounded corners, circle, line) transformed into a rudimentary car-shaped object, thereby stimulating the process of planning to solve a specific problem. The car-shaped object presents the basis of a solution to multiple problems depending upon the construction of its form.

FIG. 9 illustrates static geometric shapes (circle, square, pentagon) transformed to address different issues. One set of shapes can lead to two different forms of physiologic problem solving. Adjustment is accomplished by transforming a shape into a resulting form and then further transforming the resulting form into a final, unique geometric shape. Adaptation is accomplished by combining multiple shapes to create a transformed shape that is separate and unique from any of the contributing geometric shapes.

An embodiment of the invention provides on a display unit or as projected onto or through a surface which functions as a display unit, a singular object or many objects, at a fixed point or bounded by a region or moving or rotating in two or three-dimensional space, whether coordinated and/or synchronized or not, realized at a distance from the user in a virtual environment or digital space. The digital space is equirectangular or panoramic or wide, or realized at a distance from the user in an augmented or mixed reality or wearable environment, upon which the user gazes at shapes, images, and symbols that appear, in their partial or full field of view, which may transform their own geometric properties, and their relationship to the virtual or augmented or mixed reality or wearable environment including positioning, and graphical appearance, whether stationary or at an increased or variable speed. In addition, the visual sensory input can be augmented by any other non-visual sensory input.

The invention embodies a non-invasive, software-only brain-computer interface (hereafter “BCI”) or neural interface. “Software-only” references the control function that software executes in controlling a digital visual experience and optionally supplemental auditory stimuli, vibration, acoustics, psychoacoustics, subsonic frequencies, supersonic frequencies, hypersonic frequencies, and scent. The BCI utilizes visual, auditory, and vibratory pathways to achieve a direct form of communication from the BCI to the brain. The method of communication, or protocol, bypasses the need for conscious understanding of sensory information being sent to the brain. Notably, the invention operates without any visual or auditory language.

The invention uses the brain's ability to receive sensory input to directly inform cognitive and non-cognitive processes in the body. Most neural activity happens below the level of conscious awareness. Examples include the following: (i.) from a physiologic standpoint, people are completely unaware of the intense metabolic activity that occurs in the liver all day long; (ii.) from a disease standpoint, diabetes, usually, spends its first few decades under the radar of consciousness before manifesting physical symptoms in later life; (iiii.) from a mental illness standpoint, the manifestations of most mental illness problems in adulthood and geriatric years often have an origin in unrecognized early childhood development. In fact, the human body is designed to keep physiologic experience beneath the radar of consciousness.

The invention uses a method of brain communication that, while not limited to temporal events, can utilize timed events, such as circadian, ultradian, and infradian rhythmicity to achieve specific physiologic outcomes. The invention harnesses multiple distinct types of symbolic stimuli to guide the brain to predictable outcomes. This is achieved by utilizing a visual interface that may combine still imagery, animations, three dimensional simulations, and universal symbology. There are limitless combinations and permutations of symbology that can be harnessed to communicate specifically to the brain, and from this vast array of combinations and permutations we have identified and selected specific ways of presenting content to stimulate responses in cells, tissues, and organs throughout the body.

The invention is the only known software-based neural interface or BCI. The BCIs that exist today are hardware dependent, classified as invasive, partially invasive or non-invasive but sensor-based technologies. Software used on these brain communication devices is merely used to facilitate the operation of these devices and not designed to function independently once severed from the hardware under its control. Moreover, the use of these brain communication devices presents human health risks including, but not limited to, infection, displacement, collateral and unintended brain damage, seizures, internal bleeding, atrophy, memory loss, unpleasant sensory experiences, and cognitive fatigue. The invention does not and cannot cause any of these problems since it is a software-only invention that is platform and display agnostic and can be utilized on any available consumer (or other) hardware with a visual display.

The invention communicates information to the brain in the way that the brain natively processes external and internal environments. This includes, but is not limited to, shapes, movement, physiologic rhythms, rates, sequences, oscillations, frequencies, amplitudes. Accepted scientific protocols and expressions are integrated into the invention's software interface. Any form of hardware that outputs to, or can output to, a digital display can be used by the invention. The software serves as an external modifier to the brain's normal communication methods, bypassing the use of any human language. The user interacts with the invention in the same way that any software is accessed to perform any task. Use of the invention does not require any knowledge of underlying principles, nor does it rely upon the user's underlying intellectual capacity. In addition, use of the invention does not require the user to be in any specific state of mind, e.g. meditative, directed thinking, or specific emotional state. The user does not need to make process adjustments as these will all be handled by the invention's neural interface. The invention affects a broad spectrum of physiologic activity in the body by utilizing established methods of adjusting how the brain and peripheral body interact.

The invention does not require any clinical oversight. Furthermore, the invention does not require behavioral adjustments, psychological insight, or depend upon any established psychological practice.

The human brain assesses objects in the external and internal environments to create a hierarchy of choices, decisions, and output. The hierarchy is a list ordered by salience, which leads to decision-making. The brain's persistent ability to examine, interpret, and change the order of this hierarchy relies upon its propensity to extract information from all environments, leading to positive and negative outcomes.

Critical to hierarchical management is the brain's proficiency in extracting simple geometry from complex natural shapes found in the external and internal environments. This is mostly a passive experience for the brain, which results in a series of complex decision points that lead to a behavior, response, or proactive decisions. The interpretation of information from the external environment is not about singular geometric shapes, rather the combination of multiple geometric shapes, as well as other stimuli, reflecting the complexity of the external and internal environment. Further, it is necessary for extracted symbols to possess salience when observed, since people assign salience differently. When objects are observed by a person, salience is chosen in the context of safety, risk, desirability, and repulsion, among other cues.

The symbolic suggestion module 142 refines symbols extracted from the external and internal environments and presents visual representations of them. Symbols refined for extraction from the external environment have common meaning for all human beings, such as trees, roads, sidewalks, etc. Symbols refined for extraction from the internal environment are represented by geometric shapes, patterns, and filters that humans have established as universal interpretations, such as facial expressions, movements, gestures, and gesticulations. The digital model of the human brain in the symbolic suggestion module 142 combines shapes in a digital environment to send complex signals (such as shapes, motion, risk context) that result in specific actions on the part of the user observing the digital space. In other words, the symbolic suggestion module 142 extrapolates the method of symbolic communication utilized within the digital model of the human brain to combine and position the shapes in the digital environment displayed to the user.

The extracted objects are then organized in a hierarchy and are modified to have a predictable impact leading to a range of choices from extremes to more balanced or reasonable decision-making. In life, people rarely choose between extreme reactions (such as survival vs. risk, pleasure vs. pain, desirability vs. undesirability) and instead, typically, tend to modify their decisions in a more blended, nuanced response. The symbolic suggestion module 142 restricts the properties of symbols being displayed to a range of parameters that guide them away from choosing extreme reactions in any situation and towards a blended response that falls within a positive range of outcomes, resulting in positive neuroplastic change.

The symbolic suggestion module 142 refines symbols extracted from the external and internal environments and presents visual representations of them to yield positive outcomes.

The invention helps people make clear decisions with less extraneous interference. The invention can utilize one or more of, but not limited to, the following visual properties to enhance symbolic stimuli: movement, velocity, animation, texture, size, perspective, color, and intensity. Projecting, transforming, assembling and presenting a visual representation or properties of a symbol is performed without the use of any visual or auditory language.

An embodiment of the invention adds an auditory representation of a symbol with a full spectrum of sensory and non-sensory inputs. Embodiments of the invention can utilize one or more of, but not limited to, the following properties to enhance symbolic stimuli: sound, vibration, acoustics, psychoacoustics, amplitude, frequency, random sounds, subsonic frequencies, and supersonic or hypersonic frequencies. Projecting, transforming, assembling and presenting an auditory representation of a symbol is performed without the use of any visual or auditory language.

An embodiment of the invention aims sounds, frequency, and amplitudes to specific areas of the brain within the skull, relying upon, but not limited to, both air conduction and bone conduction.

An embodiment of the invention includes a haptic representation of a symbol with a full spectrum of sensory and non-sensory inputs. The invention can utilize one or more of, but not limited to, the following properties to enhance symbolic stimuli: vibration, tapping, and touch. Further, a vibratory or haptic feedback response may be used to evoke an emotional state or trigger brain physiologic rhythms, oscillations, and amplitudes. Projecting, transforming, assembling and presenting a haptic representation of a symbol is performed without the use of any visual or auditory language.

The content presented in the digital space can be displayed in a standalone environment or, where embedded, can dynamically interact with the flow of an environment, such as in a game program or the real world. The user can voluntarily summon the digital content or be prompted to do so if a computer program or machine learning-based or neural network-based activity or intervention determines that it is needed, whether as according to a predetermined condition in the user or a condition generated by the computer or program itself. The disclosed techniques can be used in connection with hardware for eye tracking, torso tracking, body gesture, physical or virtual button pressing, physical or virtual touch, pointing, gesturing, grabbing, finger movements, and or voice commands.

An embodiment of the technology is a display to the user when a portable head-mounted electronic device or apparatus is worn on the user's head, such as a virtual reality or augmented reality or mixed reality head-mounted device, or other such device or apparatus which may or may not retain a portable electronic device as a method of operation and communication, but which otherwise acts as a method of display to the user.

The invention implemented with a head-mounted electronic device allows the user to replace normal reality with digital reality for a specific period. In addition, the user may be presented with a full-screen, partial screen, or screen overlay, of a digital environment that may or may not be presented in a steady-state. The head-mounted device can be removed by the user at any time, such that the user does not have to remain in the digital reality if they are experiencing symptoms including, but not limited to, discomfort, eye fatigue, or stress.

The invention may be implemented with any number of immersive display devices, such as eyewear, including spectacles and contact lenses, waveguides, a holographic display device, a virtual reality display device, an augmented reality display device, and a mixed-reality display device.

The invention may be implemented as an immersive experience, such as within three-dimensional holographic displays, stereographic displays, virtual reality displays, augmented reality displays, mixed-reality displays, digital surfaces, persistence of vision displays, and robotic-assisted environments. Examples of environments suitable for immersive experiences include, but are not limited to: museums, galleries, exhibition spaces, cinemas, theatres, planetariums, theme park attractions and rides, special event hosting spaces, hotels, airport lounges, plane interiors, ship interiors, and vehicle interiors.

The digital space may be on a singular display surface but also across multiple display surfaces such as a computer, portable display screen, folding device with or without flexible display screen, mobile phone, watch, seatback or wall-mounted display, augmented reality head-up display, digital mirror device, wearables, or holographic display. Varying instances of the digital content can be shown concurrently and independently of each other across multiple display surfaces. The user may hold, place or situate in front of themselves a singular display or multiple displays while looking at and interacting with the digital content, whether by capacitive touch, eye tracking or any other method of interaction determined by the user and/or software and/or device, which either partially or in its entirety can span multiple screens and/or devices and is platform agnostic.

An embodiment of the technology is its representation in a spacecraft, space station and/or other orbital station and/or permanent infrastructure sustaining human operations on another planet and/or other astronomical body, aircraft and/or motor vehicle and/or train and/or tram and/or boat, not only on a singular display surface but also across multiple display surfaces such as a computer, portable display screen, seatback or wall-mounted display, augmented reality head-up display, digital mirror device or holographic display.

An embodiment of the technology is its representation in any environment, such as a bedroom, clinic or office, the interior of a vehicle, or an art gallery installation space, wherein a projection device or projects an original object in the surrounding area whereupon a person's gaze may be fixed at any point, whether directed to or not.

The invention provides a method for communication with the brain that to date has never existed and affects body physiology in a variety of ways. It can affect everything from energy storage and releases, and sleep performance to the ways in which the body manages metabolism including, but not limited to, blood glucose levels, organ function, coordination of bodily responses, and physiologic regulation of mood. The invention decreases human health risks, removing the risk of injury to the user, unlike invasive and partially-invasive BCIs. Clinical or health insurer oversight is not required. The invention is consumer-friendly and readily accessible.

It can be easily deployed on cheap, affordable consumer-off-the shelf hardware. The invention can be managed with minimal support.

Presently, there are no known invasive or non-invasive interventions available to purposefully regulate the brain's symbolic input via a non-invasive, software-only neural or brain-computer interface that utilizes visual, auditory, and vibratory pathways to achieve a direct form of communication from the software neural interface to the brain. All invasive BCIs use electrical stimulation, not symbology, for their input and, further, all non-invasive BCIs are sensor-based. Moreover, all current BCI devices require close and frequent physician monitoring. The only real- world methods currently available to promote brain communication are psychotherapy, behavioral therapy, and cognitive behavioral therapy. Each of these rely upon translating human language into brain communication, but the results are inconsistent, expensive, time consuming, and moderately effective at best. In addition, people can seek “self-help” through learning experiences, or meditation practices, but there is presently no method by which to direct these processes into the brain's own mechanism of effective cognitive decision-making. Lastly, there are no available systems which utilize virtual reality, augmented reality, or mixed reality to achieve the same results as the invention.

The invention can be varied or altered while accomplishing its objective as the activity involved is unrelated to the result. The user can be instructed to perform actions that have little to do with the result or otherwise perform actions without being aware of the result. There is a wide scope to vary the representation of the invention by changing any transformative property of a symbol or plurality or combination of symbols. Sensory input through peripheral and central nervous system sensors or stimulators may further vary the representation of the invention. Lastly, the invention can be accessed through a variety of software or hardware devices and be augmented by a range of observations through the application of machine learning and artificial intelligence, as well as neural learning, interfacing and processing tools.

An embodiment of the present invention relates to a computer storage product with a computer readable storage medium having computer code thereon for performing various computer-implemented operations. The media and computer code may be those specially designed and constructed for the purposes of the present invention, or they may be of the kind well known and available to those having skill in the computer software arts. Examples of computer-readable media include but are not limited to: magnetic media such as hard disks, floppy disks, and magnetic tape; optical media such as CD-ROMs, DVDs and holographic devices; magneto-optical media; and hardware devices that are specially configured to store and execute program code, such as application-specific integrated circuits (“ASICs”), programmable logic devices (“PLDs”) and ROM and RAM devices. Examples of computer code include machine code, such as produced by a compiler, and files containing higher-level code that are executed by a computer using an interpreter. For example, an embodiment of the invention may be implemented using JAVA®, C++, or other object-oriented programming language and development tools. Another embodiment of the invention may be implemented in hardwired circuitry in place of, or in combination with, machine-executable software instructions.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

1. A method, comprising:
projecting in a digital space an original object;
transforming the original object into a collection of disaggregated symbols displayed in the digital space;
assembling the collection of disaggregated symbols into an aggregated object that has qualities or features in common with the original object; and
presenting in the digital space the aggregated object augmented with auditory stimuli;
where projecting, transforming, assembling and presenting are performed without the use of any visual or auditory language.

2. The method of claim 1 wherein assembling includes changing attributes of the collection of symbols.

3. The method of claim 2 wherein changing attributes of the collection of symbols includes changing at least one of the following attributes: geometry, size, perspective, shading, texture, color, and intensity.

4. The method of claim 2 wherein changing attributes of the collection of symbols includes altering at least one symbol through movement, growth, contraction, and position.

5. The method of claim 1 further comprising augmenting the digital space with at least one of vibration, acoustics, psychoacoustics, subsonic frequencies, supersonic frequencies, hypersonic frequencies, and scent.

6. The method of claim 1 implemented on a head-mounted device.

7. The method of claim 1 implemented on an immersive device.

8. The method of claim 1 implemented in an immersive experience.

9. The method of claim 1 implemented on or via a brain computer interface.

10. The method of claim 1 implemented on a display device.

11. The method of claim 1 implemented on multiple display devices.

12. The method of claim 1 implemented on a circular display device.

13. The method of claim 1 implemented on a projection display device.

14. The method of claim 1 wherein the digital space is two-dimensional.

15. The method of claim 1 wherein the digital space is three-dimensional.

16. The method of claim 1 wherein the digital space is augmented with analog audio.

17. The method of claim 1 wherein the digital space is augmented with digital audio.

18. The method of claim 1 wherein the digital space is augmented with content from an artificial intelligence agent.

19. The method of claim 1 wherein the digital space is augmented with content from a machine learning model.

20. The method of claim 1 implemented on a holographic display.
